(19) United States
(12) Patent Application Publication
(10) Pub. No.: US 2023/0257798 A1
(43) Pub. Date: Aug. 17, 2023

(54) METHOD FOR PROVIDING PREPARATION FOR DETECTING TARGET NUCLEIC ACID SEQUENCE IN SPECIMEN

(71) Applicant: SEEGENE, INC., Seoul (KR)

(72) Inventors: Yun Jee KIM, Seoul (KR); Ju hee BAE, Seoul (KR); So dam JIN, Guri-si, Gyeonggi-do (KR); Sanghun GWON, Seoul (KR)

(73) Assignee: SEEGENE, INC., Seoul (KR)

(21) Appl. No.: 18/011,678

(22) PCT Filed: Jun. 21, 2021
(86) PCT No.: PCT/KR2021/007762

§ 371 Date: Dec. 20, 2022

(30) Foreign Application Priority Data
Jun. 22, 2020 (KR) 10-2020-0076052

(51) Int. Cl. C12Q 1/6806; C12Q 1/70

(52) U.S. Cl. CPC C12Q 1/6806; C12Q 1/70

(57) ABSTRACT

Disclosed herein is a method for providing a preparation for detecting a target nucleic acid sequence in a specimen. According to an embodiment, conventional nucleic acid extraction processes performed in many steps can be omitted, whereby the shortage of nucleic acid extraction reagents can be solved and a preparation for detecting target nucleic acid sequence in a specimen can be supplied in an inexpensive and simple manner.

### TECHNICAL FIELD

The present disclosure relates to a method for providing a preparation for detection of a target nucleic acid sequence in a specimen.

### BACKGROUND ART

Molecular diagnostics is one of the fields that are rapidly growing in the in vitro diagnosis markets for early diagnosis of diseases. Inter alia, methods using nucleic acids are advantageously used to diagnose causative genetic factors such as infections with viruses, bacteria, etc., on the basis of the high specificity and sensitivity thereof. Most of the diagnosis methods using nucleic acids include amplification of target nucleic acids (e.g., viral or bacterial nucleic acids). Polymerase chain reaction (PCR), which is representative of nucleic acid amplification methods, includes repeated cycles of the denaturation of double-stranded DNA, the annealing of oligonucleotide primers to a DNA template, and the extension of the primers by DNA polymerase (Mullis et al., U.S. Pat. Nos. 4,683,195, 4,683,202, and 4,800,159; and Saiki et al., (1985) Science 230, 1350-1354).

Suggested for nucleic acid amplification has been a variety of methods including LCR (Ligase Chain Reaction), SDA (Strand Displacement Amplification), NASBA (Nucleic Acid Sequence-Based Amplification), TMA (Transcription Mediated Amplification), RPA (Recombinase polymerase amplification), LAMP (Loop-mediated isothermal amplification), and RCA (Rolling-Circle Amplification).

With the pandemic onset of severe acute respiratory syndrome coronavirus 2 (SARS-CoV-2), reverse transcription-PCR (RT-PCR) diagnosis kits for detecting SARS-CoV-2 have recently attracted great attention. As a rule, coronavirus diagnosis based on an RT-PCR diagnosis kit requires processes of sampling a specimen (e.g., a nasopharyngeal swab and/or an oropharyngeal swab, etc.), storing the specimen in a universal transport medium, and extracting a nucleic acid (e.g., RNA) before RT-PCR.

In conventional nucleic acid extraction methods, a sample containing cells is treated with a potent surfactant such as SDS, or a kit taking advantage of silica or glass fibers binding specifically to nucleic acids is used.

However, the pandemic of SARS-CoV-2 has triggered a rapid increase in demand on nucleic acid extraction reagents in addition to diagnosis kits, deepening a lack of nucleic acid extraction reagents. As a result, the lack of extraction reagents results in a serious bottleneck effect in coronavirus diagnosis.

In order to solve the lack of extraction reagents, various attempts have been made. There is a need for a novel method for providing a preparation for detection of a target nucleic acid sequence in a specimen, without using nucleic acid extraction reagents.

Numerous documents and patent documents are referred to and cited throughout this specification. The disclosures of the cited documents and patents are incorporated herein by reference in their entireties to more clearly describe the state of the art to which the present disclosure pertains, and the content of the present disclosure.

### SUMMARY OF THE INVENTION

Intensive and thorough research has been conducted in order to develop a novel method for providing preparations for detection of target nucleic acids in an inexpensive and simplified manner, with the aim of solving the supply shortage of nucleic acid extraction reagents.

As a result, it was found that incubation of a specimen at 95° C. to 100° C. for 1 to 25 minutes allows the provision of preparations for detection of target nucleic acids in specimens in a more inexpensive and simplified manner while solving the supply shortage of nucleic acid extraction reagents without nucleic acid degradation.

Therefore, an aspect of the present disclosure is to provide a method for providing a preparation for detection of a target nucleotide sequence in a specimen.

Other objects and advantages of the present disclosure are more apparent from the following detailed description together with the appended claims.

### DETAILED DESCRIPTION OF THE INVENTION

Provided according to an aspect of the present disclosure is a method for providing a preparation for detection of a target nucleic acid sequence in a specimen.

An embodiment of the present disclosure provides a method for providing a preparation for detection of a target nucleic acid sequence in a specimen, the method comprising the steps of:

(a) incubating a specimen stored in a transport medium at 95° C. to 100° C. for 1 minute to 25 minutes to provide preparation, wherein the specimen is lysed by the incubation and has viscosity; and

(b) mixing the preparation with a composition for detecting a target nucleic acid.

Hereinafter, the present disclosure will be described in more detail as follows:

Step (a): Provision of Preparation for Detecting Target Nucleic Acid

The present disclosure provides preparation for detection of a target nucleic acid.

According to an embodiment of the present disclosure, a specimen stored in a transport medium may be lysed by incubation at 95° C. to 100° C. for 1 minute to 25 minutes.

As used herein, the term “specimen” means a sample containing a target nucleic acid or a sample considered to contain a target nucleic acid. The specimen can be obtained from a subject such as a human or an animal.

As used herein, the term “nucleic acid”, “nucleic acid sequence” or “nucleic acid molecule” refer to a single-stranded or double-stranded deoxyribonucleotide or ribonucleotide polymer which may contain derivatives of naturally occurring nucleotides, non-naturally occurring nucleotides, or modified nucleotides, which can function in the same manner as naturally occurring nucleotides.

The terms “target nucleic acid”, “target nucleic acid sequence”, or “target sequence”, as used herein, refers to a nucleic acid sequence to be detected, which is intended to be annealed or hybridized with a probe or primer in an annealing, hybridizing, or amplification condition.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. The primers need to be sufficiently long to prime the synthesis of extension products in the presence of the agent for polymerization. The appropriate length of the primers depends on several factors, such as temperature, field of application, and sources of primers.

In an embodiment, the oligonucleotide may be 5 to 1,000 bp, 5 to 900 bp, 5 to 800 bp, 5 to 700 bp, 5 to 600 bp, 5 to 500 bp, 5 to 400 bp, 5 to 300 bp, 5 to 200 bp, 5 to 150 bp, 5 to 100 bp, 5 to 90 bp, 5 to 80 bp, 5 to 70 bp, 5 to 60 bp, 5 to 50 bp, 5 to 40 bp, 5 to 30 bp, 5 to 20 bp, 5 to 10 bp, 10 to 1,000 bp, 10 to 900 bp, 10 to 800 bp, 10 to 700 bp, 10 to 600 bp, 10 to 500 bp, 10 to 400 bp, 10 to 300 bp, 10 to 200 bp, 10 to 150 bp, 10 to 100 bp, 10 to 90 bp, 10 to 80 bp, 10 to 70 bp, 10 to 60 bp, 10 to 50 bp, 10 to 40 bp, 10 to 30 bp, 10 to 20 bp, 15 to 1,000 bp, 15 to 900 bp, 15 to 800 bp, 15 to 700 bp, 15 to 600 bp, 15 to 500 bp, 15 to 400 bp, 15 to 300 bp, 15 to 200 bp, 15 to 150 bp, 15 to 100 bp, 15 to 90 bp, 15 to 80 bp, 15 to 70 bp, 15 to 60 bp, 15 to 50 bp, 15 to 40 bp, or 15 to 30 bp, but is not limited thereto.

As used herein, the term “probe” refers to a single-stranded nucleic acid molecule including a region or regions complementary substantially to a target nucleic acid sequence. According to an embodiment, the probe may be “blocked” at the 3′-terminal thereof and thus is prevented from extending therefrom. The blocking may be achieved in accordance with conventional methods. For instance, the blocking may be performed by adding to the 3′-hydroxyl group of the last nucleotide a chemical moiety such as biotin, labels, a phosphate group, alkyl group, non-nucleotide linker, phosphorothioate or alkane-diol. Alternatively, the blocking may be carried out by removing the 3′-hydroxyl group of the last nucleotide or using a nucleotide with no 3′-hydroxyl group such as dideoxynucleotide. The primer or the probe may be a single strand. The primer or the probe may include deoxyribonucleotides, ribonucleotides, or a combination thereof. The primer or probe used in the present disclosure may include naturally occurring dNMPs (e.g., dAMP, dGMP, dCMP, and dTMP), modified nucleotides, or non-naturally occurring nucleotides.

As used herein, “annealing” or “priming” refers to the apposition of an oligonucleotide or nucleic acid to a template nucleic acid, whereby the apposition enables polymerase to polymerize nucleotides into a nucleic acid molecule which is complementary to the template nucleic acid or a portion thereof.

As used herein, the term, “hybridization” refers to the formation of a double-stranded nucleic acid from two single-stranded polynucleotides through non-covalent binding between complementary nucleotide sequences under predetermined hybridization conditions or stringent conditions.

In an embodiment of the present disclosure, the specimen may be a viscous specimen, for example, a swab, saliva, sputum, an aspirate, bronchoalveolar lavage (BAL), a gargle sample, or blood, but without limitations thereto.

In an embodiment, the specimen may be a swab, saliva, or a combination thereof.

In an embodiment, the specimen may be a swab, for example, a nasopharyngeal swab, an oropharyngeal swab, a saliva swab, a genital swab, a rectal swab, or a combination thereof, but without limitations thereto.

According to an embodiment, the specimen may be a nasopharyngeal swab, an oropharyngeal swab, a saliva swab, a genital swab, or a rectal swab.

According to another embodiment, the specimen may be a combination of two of a nasopharyngeal swab, an oropharyngeal swab, a saliva swab, a genital swab, and a rectal swab. For example, the specimen may be a combination of a nasopharyngeal swab and an oropharyngeal swab.

According to a further embodiment, the specimen may be a combination of three of a nasopharyngeal swab, an oropharyngeal swab, a saliva swab, a genital swab, and a rectal swab. For example, the specimen may be a combination of a nasopharyngeal swab, an oropharyngeal swab and a saliva swab.

According to another embodiment, the specimen may be saliva.

According to another embodiment, the specimen may be a gargle sample. For example, the specimen may be an oral gargle sample, a throat gargle sample, or a combination thereof.

In an embodiment, the target nucleic acid may be a nucleic acid from a human, an animal, a plant, or a microorganism. The microorganism may be a fungus, a protozoa, a bacterium, a virus, or an alga.

In an embodiment, the target nucleic acid may be a viral nucleic acid. In detail, the target nucleic acid may be an RNA virus nucleic acid.

In an embodiment, the target nucleic acid may be a respiratory virus nucleic acid. For example, the target nucleic acid may include an influenza virus nucleic acid, a respiratory syncytial virus (RSV) nucleic acid, an adenovirus nucleic acid, an enterovirus nucleic acid, a parainfluenza virus nucleic acid, a metapneumovirus (MPV) nucleic acid, a bocavirus nucleic acid, a rhinovirus nucleic acid, and/or a coronavirus nucleic acid.

According to a particular embodiment, the target nucleic acid may be severe acute respiratory syndrome coronavirus 2 (SARS-CoV-2) nucleic acid.

In an embodiment of the present disclosure, the swab may be obtained using various sample collection devices. Representative examples of the devices for collecting a swab specimen include FLOQSWABS®, CLASSIQSWABS™ Dry Swabs, etc.

According to an embodiment, when the specimen includes a swab, the swab specimen may be collected using a specimen sampling rod such like cotton swab. For instance, a specimen for detection of respiratory virus can be obtained by applying a rod to a region from which much secretions can be obtained, for example, the inner wall of the nasopharynx or oropharynx, and rotating the applied rod to take secretions from the mucous membrane of the inner wall in the nasopharynx or oropharynx. The swab rod having a specimen absorbed thereto may be stored in a transport medium that allows the viruses, bacteria, etc. in the specimen to retain their original states for a long period of time without undergoing death or excessive proliferation, or the specimen absorbed onto the swab rod may be dissolved in such a transport medium for storage.

According to an embodiment, the transport medium may be a preservative transport medium.

In an embodiment, water or TE buffer may serve as a transport medium for saliva or sputum.

In an embodiment, saliva or sputum may be added into a transport medium before transport or may be transported as it is, and then mixed with a transport medium before incubation.

In an embodiment, a washing fluid and a gargle solution may serve as transport media for bronchoalveolar lavage (BAL) and a gargle sample, respectively.

In an embodiment, the bronchoalveolar lavage or the gargle sample may be added into a transport medium before transport, or may be transported as it is and then mixed with a transport medium before incubation.

As used herein, the term “transport medium” refers to a medium that allows viruses, bacteria, etc. in a specimen collected for diagnostic test from an subject (e.g., a patient) to retain their original states for a long period of time without undergoing death or excessive proliferation, and includes a solution (e.g., water or buffer) used for diluting a specimen.

So long as it allows viruses, bacteria, etc. in a specimen collected for diagnostic test from a patient to retain their original states for a long period of time without undergoing death and excessive proliferation and to be delivered to an inspect agency, any medium may be used as a transport medium. For example, the transport medium may be a universal transport medium (UTM), a viral transport medium (VTM), or a clinical virus transport medium (CTM) from Noble Bio, which can transport viruses or bacteria alive.

According to an embodiment, the transport medium does not contain any material for lysis of a specimen.

The term “material for lysis”, as used herein, refers to an ingredient used to lyse a specimen (e.g., cells) in a chemical cell lysis manner and may include, for example, an acid, a base, a detergent, a solvent, a chaotropic substance, etc.

According to an embodiment, the transport medium may be a liquid medium.

According to an embodiment of the present disclosure, the specimen stored in the transport medium may be incubated at 95° C. to 100° C. for 1 minute to 25 minutes to lyse the specimen.

According to an embodiment, the transport medium containing a specimen is aliquoted into containers before incubation. For example, the transport medium containing a specimen may be aliquoted in an amount of 2 μl to 1,000 μl, 2 μl to 900 μl, 2 μl to 800 μl, 2 μl to 700 μl, 2 μl to 600 μl, 2 μl to 500 μl, 2 μl to 400 μl, 2 μl to 300 μl, or 2 μl to 100 μl, particularly in an amount of 5 μl to 100 μl, 5 μl to 70 μl, or 5 μl to 50 μl, and more particularly in an amount of 10 μl to 40 μl into containers.

According to another embodiment, without separate division into aliquots, the transport medium containing a specimen may be incubated in the initial container where the sampled specimen has been first stored. From the incubated container, the transport medium may be aliquoted and mixed with a composition for detecting a target nucleic acid.

According to an embodiment, the specimen stored in the transport medium may be incubated without dilution.

According to another embodiment, the specimen stored in the transport medium may be diluted with water or a buffer before incubation. By way of example, when incubation is carried out without separate division into aliquots, the transport medium containing a specimen may be diluted with water or a buffer. When incubation is carried out after the transport medium containing a specimen is aliquoted into containers, the containers may be filled in advance with water or a buffer for dilution or may be added with water or a buffer subsequently to the fractional division.

The dilution can weaken the inhibitory activity of a possible reaction inhibitor in the transport medium when the specimen is mixed with the composition for detecting target nucleic acid.

In an embodiment, a highly viscous specimen (e.g., saliva or sputum) may be diluted to reduce the viscosity before incubation, thereby making it easy to dissolve the specimen.

The water for use in dilution may be, for example, purified water, nuclease-free-water (i.e., RNase-free water), or distilled water.

The buffer for use in dilution may be, for example, a TE buffer.

According to an embodiment, the specimen stored in a transport medium may be diluted 1 to 10 fold, particularly, 2 to 8 fold, 2 to 7 fold, 2 to 6 fold, 2 to 5 fold, or 2 to 4 fold, and more particularly 2 to 4 fold. For example, when the specimen stored in a transport medium is 2-fold diluted, the transport medium containing the specimen may be 1:1 mixed with water (or a buffer).

As used herein, the term “specimen lysis” is intended to encompass the lysis of membranes or walls of cells, bacteria, viruses, etc. in the specimen (e.g., swab, saliva, etc.).

As used herein, the term “specimen viscosity” refers to the viscosity of a sampled specimen itself (e.g., the viscosity of rhinorrhea itself taken on a swab rod or the viscosity of sputum itself) or the viscosity of the solution in which the sampled specimen is stored (e.g., a transport medium in which the rhinorrhea taken on a swab rod is dissolved).

According to an embodiment, the incubation may be conducted at 95° C. or higher, 96° C. or higher, 97° C. or higher, 98° C. or higher, or 99° C. or higher. In an embodiment, the incubation temperature is 100° C. or lower or 99° C. or lower.

According to an embodiment, the incubation temperature ranges from 97° C. to 100° C., from 97° C. to 99° C., from 97° C. to 98° C., from 98° C. to 100° C., or from 98° C. to 99° C.

According to an embodiment, the incubation temperature is 98° C.

The incubation temperature may be set such that a lysate of a specimen can be obtained.

According to an embodiment, the incubation may be conducted for 1 minute or longer, 2 minutes or longer, or 3 minutes or longer. According to an embodiment, the incubation may be conducted for 25 minutes or shorter, 23 minutes or shorter, or 20 minutes or shorter.

According to an embodiment, the incubation may be conducted for 1 minute to 25 minutes, 1 minute to 20 minutes, 2 minutes to 25 minutes, 2 minutes to 20 minutes, 3 minutes to 25 minutes, or 3 minutes to 20 minutes.

The incubation time may be set such that the target nucleic acid in the specimen is prevented from being degraded at the incubation temperature.

For example, a specimen with relatively high viscosity is incubated for a longer period of time than a specimen with relatively low viscosity. In contrast, incubation is conducted for a shorter time on a specimen with a relatively low viscosity than a specimen with a relatively high viscosity, thereby preventing the degradation of the nucleic acid.

According to an embodiment, when the specimen is a swab, the incubation may be conducted for 2 minutes to 4 minutes.

In an embodiment, when the specimen is a swab, the incubation may be conducted for 3 minutes.

According to an embodiment, when the specimen is saliva, sputum, a gargle sample, an aspirate, bronchoalveolar lavage (BAL), or blood, the incubation may be conducted for 18 minutes to 22 minutes.

In an embodiment, when the specimen is saliva, sputum, a gargle sample, an aspirate, bronchoalveolar lavage (BAL), or blood, the incubation may be conducted for 20 minutes.

In an embodiment, a highly viscous specimen, for example, saliva or sputum, may be treated with proteinase K to lower the viscosity of the specimen and then incubated to make the lysis of the specimen easy.

According to an embodiment, the use of a separate lysis material is not required because the membranes or walls of cells, bacteria, viruses, etc. in a specimen can be lysed only with the selected temperature and incubation time.

The incubation temperature and time may be selected variously as long as it allows the lysis of membranes or walls of cells, bacteria, viruses, etc. and prevents the degradation of a target nucleic acid present in a specimen.

In an embodiment, the incubation may be conducted at 95° C. to 100° C. for 1 minute to 25 minutes.

In an embodiment, the incubation may be conducted at 95° C. to 100° C. for 2 minutes to 20 minutes.

In an embodiment, the incubation may be conducted at 97° C. to 100° C. for 2 minutes to 25 minutes.

In an embodiment, the incubation may be conducted at 97° C. to 100° C. for 2 minutes to 20 minutes.

In an embodiment, the incubation may be conducted at 98° C. to 100° C. for 2 minutes to 25 minutes.

In an embodiment, the incubation may be conducted at 98° C. to 100° C. for 2 minutes to 20 minutes.

In an embodiment, the incubation may be conducted at 98° C. for 2 minutes to 25 minutes.

In an embodiment, the incubation may be conducted at 98° C. for 2 minutes to 20 minutes.

In an embodiment, when the specimen is a swab, the incubation may be conducted at 97° C. to 100° C. for 2 minutes to 4 minutes, particularly at 98° C. for 3 minutes.

In an embodiment, when the specimen is saliva, sputum, a gargle sample, an aspirate, bronchoalveolar lavage (BAL), or blood, the incubation may be conducted at 97° C. to 100° C. for 18 minutes to 22 minutes and particularly at 98° C. for 20 minutes.

To the knowledge of the present inventors, the incubation temperature and time selected in the present disclosure is a condition in which a lysate of a specimen can be effectively obtained while the degradation of a target nucleic acid can be prevented, without a separate nucleic acid extraction process. Furthermore, it is a condition in which a target nucleic acid can optimally work as a template. Particularly, when the target nucleic acid is an RNA, the incubation temperature and time set forth in the present disclosure was found to be a condition in which cDNA can be effectively prepared from the target RNA.

In an embodiment, the incubation may be carried out on a volume of 2 μl to 4,000 μl, 2 μl to 3,000 μl, 2 μl to 2,000 μl, 2 μl to 1,000 μl, 2 μl to 900 μl, 2 μl to 800 μl, 2 μl to 700 μl, 2 μl to 600 μl, 2 μl to 500 μl, 2 μl to 400 μl, 2 μl to 300 μl, 2 μl to 200 μl, 2 μl to 100 μl, 50 μl to 4,000 μl, 50 μl to 3,000 μl, 50 μl to 2,000 μl, 50 μl to 1,000 μl, 50 μl to 900 μl, 50 μl to 800 μl, 50 μl to 700 μl, 50 μl to 600 μl, 50 μl to 500 μl, 50 μl to 400 μl, 50 μl to 300 μl, 50 μl to 200 μl, 50 μl to 100 μl, 100 μl to 4,000 μl, 100 μl to 3,000 μl, 100 μl to 2,000 μl, 100 μl to 1,000 μl, 100 μl to 900 μl, 100 μl to 800 μl, 100 μl to 700 μl, 100 μl to 600 μl, 100 μl to 500 μl, 100 μl to 400 μl, 100 μl to 300 μl, 100 μl to 200 μl, 200 μl to 4,000 μl, 200 μl to 3,000 μl, 200 μl to 2,000 μl, 200 μl to 1,000 μl, 200 μl to 900 μl, 200 μl to 800 μl, 200 μl to 700 μl, 200 μl to 600 μl, 200 μl to 500 μl, 200 μl to 400 μl, or 200 μl to 300 μl, particularly on a volume of 5 μl to 100 μl, 5 μl to 70 μl, 10 μl to 100 μl, or 10 μl to 70 μl, and more particularly on a volume of 10 μl to 60 μl, without limitations thereto.

The volume may be the quantity of the specimen-containing transport medium which is or is not diluted.

In an embodiment, the division to aliquots can be carried out using an automated dispenser system (e.g., Liquid Handler). Using a liquid handler, for example, the transport medium may be aliquoted at a flow rate of 10 μl/s to 400 μl/s, particularly at a flow rate of 10 μl/s to 300 μl/s, 10 μl/s to 200 μl/s, 10 μl/s to 100 μl/s, 20 μl/s to 100 μl/s, 20 μl/s to 80 μl/s, 20 μl/s to 70 μl/s, 30 μl/s to 80 μl/s, 30 μl/s to 70 μl/s, or 30 μl/s to 60 μl/s, without limitations thereto.

Step (b): Mixing of Preparation and Target Nucleic Acid Detecting Composition

According to the present disclosure, the preparation obtained by incubation is mixed with the target nucleic acid detecting composition.

According to an embodiment, the preparation may be cooled to 2° C. to 10° C. and mixed with the target nucleic acid detecting composition. In detail, the preparation may be cooled to 4° C. to 7° C. and particularly to 4° C.

In an embodiment, the preparation may be aliquoted and mixed with a target nucleic acid detecting composition.

According to an embodiment, the preparation may be aliquoted into a volume of 1 μl to 100 μl, 1 μl to 90 μl, 1 μl to 80 μl, 1 μl to 70 μl, 1 μl to 60 μl, 1 μl to 50 μl, 1 μl to 40 μl, 1 μl to 30 μl, 1 μl to 20 μl, 1 μl to 10 μl, 3 μl to 100 μl, 3 μl to 90 μl, 3 μl to 80 μl, 3 μl to 70 μl, 3 μl to 60 μl, 3 μl to 50 μl, 3 μl to 40 μl, 3 μl to 30 μl, 3 μl to 20 μl, or 3 μl to 10 μl, particularly into a volume of 1 μl to 10 μl, 1 μl to 7 μl, 3 μl to 10 μl, or 3 μl to 7 μl, and more particularly into a volume of 3 μl to 5 μl.

According to an embodiment, the preparation may be mixed at a ratio of 1:1 to 1:20, particularly 1:2 to 1:10, and more particularly 1:2 to 1:4 with the target nucleic acid detecting composition.

In an embodiment, the preparation may be mixed with the target nucleic acid detecting composition, without a separate nucleic acid extraction process.

As the recent pandemic breakout of SARS-CoV-2 has rapidly increased a demand on nucleic acid extraction reagents and diagnosis kit reagents, reagents used in a nucleic acid extraction step, for example, proteinase K, are difficult to supply.

The method according to the present disclosure can be performed without using separate nucleic acid extraction reagents. Particularly, when the specimen is a swab, the method can be performed without using proteinase K. Therefore, the preparation or mixture does not contain proteinase K when the specimen is a swab.

According to an embodiment, the preparation may be mixed with the target nucleic acid detecting composition without being diluted.

In an embodiment, the transport medium contained in the preparation that is mixed with the target nucleic acid detecting composition may be identical in concentration to the transport medium in which the sampled specimen is stored initially.

According to an embodiment, the preparation may be diluted with water or a buffer before being mixed with the target nucleic acid detecting composition.

In an embodiment, when the transport medium in which the specimen is stored is incubated without dilution, the preparation may be diluted with water or a buffer.

In an embodiment, the transport medium contained in the preparation that is mixed with the target nucleic acid detecting composition may be in a diluted state, compared to the transport medium in which the specimen is stored.

The water for use in dilution may be, for example, purified water, nuclease-free-water (e.g., RNase-free water), or distilled water.

The buffer for use in dilution may be, for example, a TE buffer.

According to an embodiment, the preparation may be diluted 1 to 10 fold, particularly, 2 to 8 fold, 2 to 7 fold, 2 to 6 fold, 2 to 5 fold, or 2 to 4 fold, and more particularly 2 to 4 fold. For example, when the preparation is 2-fold diluted, the preparation may be 1:1 mixed with water (or a buffer).

The dilution can weaken the inhibitory activity of a possible reaction inhibitor in the transport medium when the preparation is mixed with the target nucleic acid detecting composition.

In an embodiment, the preparation may be diluted while being mixed with the target nucleic acid detecting composition. For example, the container in which the preparation and the composition are mixed may be filled in advance with water or a buffer for dilution, or the preparation may be aliquoted into a container, diluted by adding water or a buffer, and then mixed with the composition. In another embodiment, the preparation may be mixed with the composition and then diluted with water or a buffer.

The mixture provided according to the present disclosure can be used in a target nucleic acid detection reaction.

According to an embodiment, the target nucleic acid detection reaction may include a target nucleic acid amplification reaction.

According to another embodiment, the target nucleic acid detection reaction may include a multiple target nucleic acid sequence amplification reaction.

As used herein, the term “multiple target nucleic acid sequence amplification reaction” refers to a reaction in which two or more nucleic acid sequences are targeted and amplified. In the multiple target nucleic acid sequence amplification reaction, two or more nucleic acid sequences are amplified together by a single reaction. For example, multiple target nucleic acid sequence amplification reaction may amplify 2 or more, 3 or more, 4 or more, 5 or more, 6 or more, 7 or more, 8 or more, 9 or more, 10 or more, or 15 or more, target nucleic acid sequences together in a single reaction.

In an embodiment, the amplification of a target nucleic acid may be achieved by polymerase chain reaction (PCR).

Polymerase chain reaction is widely used in the art so as to amplify a target nucleic acid and includes repeated cycles of denaturing a target nucleic acid sequence, annealing (hybridizing) between the target nucleic acid sequence and a primer, and extending the primer (U.S. Pat. Nos. 4,683,195, 4,683,202, and 4,800,159; and Saiki et al., (1985) Science 230, 1350-1354).

For a double-stranded target nucleic acid, the double strand is preferably prepared into single strand forms or a partially single-stranded form. Examples of a method for separating a double strand include, but are not limited to, heating, alkali, formamide, urea and glycoxal treatment, enzymatic treatment (e.g., helicase action), and binding proteins. For instance, strand separation can be achieved by heating at a temperature ranging from 80° C. to 105° C. With respect to general methods for accomplishing this treatment, reference may be made to Joseph Sambrook, et al., Molecular Cloning, A Laboratory Manual, Cold Spring Harbor Laboratory Press, Cold Spring Harbor, N.Y. (2001).

The annealing of a primer to a target sequence may be carried out under suitable hybridization conditions routinely determined by optimization procedures. Conditions such as temperatures, concentrations of components, hybridization and washing times, buffer components, and their pH and ionic strength may vary depending on various factors, including the length and GC content of oligonucleotides (primers) and the target nucleotide sequence. For details of hybridization, reference may be made to Joseph Sambrook et. al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

The primer annealed to the target sequence is extended by a template-dependent polymerase, including “Klenow” fragment of E. coli DNA polymerase I, a thermostable DNA polymerase, and bacteriophage T7 DNA polymerase. In an embodiment of the present disclosure, the template-dependent polymerase is a thermostable DNA polymerase obtained from a variety of bacterial species.

When a polymerization reaction is being conducted, the components required for such reaction may be provided in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, and dATP, dCTP, dGTP, and dTTP in sufficient quantity to support the degree of the extension desired.

According to another embodiment, alternative examples of the method for amplification of a target nucleic acid sequence include LCR (Ligase Chain Reaction), SDA (Strand Displacement Amplification), NASBA (Nucleic Acid Sequence-Based Amplification), TMA (Transcription Mediated Amplification), RPA (Recombinase polymerase amplification), LAMP (Loop-mediated isothermal amplification), and RCA (Rolling-Circle Amplification), but are not limited thereto.

In an embodiment, the target nucleic acid detection reaction may detect or quantify a target nucleic acid sequence in a post-PCR detection manner or real-time detection manner.

In an exemplary embodiment, the detection or quantification is achieved by detecting a detection signal generated depending on the presence of a target nucleic acid sequence.

The real-time detection approach may be carried out using a non-specific fluorescent dye that is non-specifically intercalated into a duplex, which is an amplicon of the target nucleic acid sequence.

Additionally, the real-time detection approach may utilize a labeled probe that specifically hybridizes with a target nucleic acid sequence. Examples of the approach include a molecular beacon method that uses a dual-labeled probe with a hairpin structure (Tyagi et al, Nature Biotechnology v. 14 Mar. 1996), a hybridization probe method that uses two probes singly labeled with a donor or an acceptor (Bernad et al, 147-148 Clin Chem 2000; 46), a Lux method that uses a single-labeled oligonucleotides (U.S. Pat. No. 7,537,886), and a TaqMan method that uses hybridization of a dual-labeled probe and its cleavage by 5′-nuclease activity of DNA polymerase as well as (U.S. Pat. Nos. 5,210,015 and 5,538,848), but are not limited thereto.

Alternatively, real-time detection may be carried out using a duplex formed depending on the presence of a target nucleic acid sequence. The duplex formed depending on the presence of a target nucleic acid sequence is not an amplicon of the target sequence per se formed by the amplification reaction, but a duplex the amount of which is increased in proportion to the amplification of the target nucleic acid sequence. The duplex formed depending on the presence of the target nucleic acid sequence may be obtained in accordance with various methods, for example, the PTOCE (PTO Cleavage and Extension) method disclosed in WO 2012/096523, which is herein incorporated by reference in its entirety.

In addition, the real-time detection of a target in the present disclosure may be achieved using a method for detecting one or more target nucleic acid sequences with only a single-type label by using signal detection at different temperatures. For this content, reference may be made to WO 2015/147370, WO 2015/147377, WO 2015/147382, and WO 2015/147412, which are all incorporated herein by reference in their entireties.

The post-PCR detection approach is a method for detection of an amplicon after amplification of a nucleic acid. Examples of the post-PCR detection method include, but are not limited to, separation of amplicons by size (generally performed by gel electrophoresis) or separation by immobilization of amplicons.

As alternatives for post-PCR detection approach, the post-PCR melting assay has been suggested in which fluorescent intensities are monitored while the temperature is increased or decreasing within a certain range after amplification of the target nucleic acid sequence and then amplicons are detected using the melting profile (U.S. Pat. Nos. 5,871,908 and 6,174,670 and WO 2012/096523).

A real-time PCR assay employing a standard quantification curve and Cₜ (threshold cycle) values may be used to quantify target nucleic acid sequences. The post-PCR melting assay using heights or areas of melting peaks can be applied to quantification.

According to an embodiment, the target nucleic acid detection reaction may detect the presence of a target nucleic acid sequence by real-time PCR.

According to an embodiment, when the target nucleic acid is an RNA, the target nucleic acid detection reaction may include a reverse-transcription step prior to the target nucleic acid amplification reaction, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988).

Reverse transcription-polymerase chain reaction (RT-PCR) is a technique combining reverse transcription of RNA to cDNA by a reverse transcriptase and amplification of target DNA fragments with the cDNA serving as a template.

As used herein, the term “reverse transcriptase” is an RNA-dependent DNA polymerase that synthesizes complementary DNA from an RNA template.

In the present disclosure, the reverse transcriptase may be originated from various sources. Examples of the reverse transcriptase include, but are not limited to, Avian Myeloblastosis Virus-derived Reverse Transcriptase (AMV RTase), Murine Leukemia Virus-derived Reverse Transcriptase (MuLV RTase), and Rous-Associated Virus 2 Reverse Transcriptase (RAV-2 RTase).

Reverse transcriptase requires a primer for synthesizing cDNA from an RNA template. There are largely three types of primers for use in reverse transcriptase reaction: (i) an oligo dT primer that is annealed to poly A tail in mRNA to allow for the synthesis of cDNA from the 3′ terminus; (ii) a random primer that is made of random nucleotides 6-9 nt long and allows cDNA synthesis to start at any site of RNA; and (iii) a target-specific primer for synthesizing only a target cDNA.

In an embodiment, the target nucleic acid detecting composition may comprise a reverse transcription composition.

In an embodiment, the reverse transcription composition may contain a reverse transcriptase and/or a reverse transcription primer.

In an embodiment, the reverse transcription primer may be an oligo dT-primer, a random primer, or a target-specific primer.

According to an embodiment, the reverse transcription primer may be a target-specific primer.

In an embodiment, the target-specific primer may be used for cDNA synthesis in reverse transcription and, in turn, may form a pair with a different primer (e.g., forward primer or reverse primer) in the target nucleic acid sequence amplification reaction, thereby serving as a primer pair for amplification of the cDNA thus synthesized.

In the present disclosure, the reverse transcription composition may optionally contain reagents necessary for reverse transcription, such as a buffer, dNTPs (deoxyribonucleotide triphosphates), and so on. Optimal amounts of reagents used in a specific reaction can be easily determined by a person skilled in the art who understands the advantages of the present disclosure. Components of the reverse transcription composition may exist in respective individual containers or may be contained in combination in a single container.

According to an embodiment, reverse transcription may be carried out using the mixture.

According to an embodiment, the target nucleic acid detection reaction may include RT-PCR (Reverse Transcription PCR) and, more particularly, one-step RT-PCR.

In an embodiment, the target nucleic acid detecting composition may be provided as a composition for synthesizing cDNA from RNA and a composition for amplifying and detecting the cDNA in respective separate containers or as a mixture in a single container.

According to an embodiment, the target nucleic acid detecting composition may contain an oligonucleotide (e.g., a primer or a probe) for amplification and/or detection of the target nucleic acid.

According to an embodiment, the target nucleic acid detecting composition may contain an internal control nucleic acid sequence, and an oligonucleotide (e.g., a primer or a probe) for amplification and/or detection of the internal control nucleic acid sequence.

According to an embodiment, the target nucleic acid detection reaction may be conducted together with the detection of the internal control.

The internal control may be an endogenous internal control contained from the specimen sampling step or may be an exogenous internal control added after the specimen sampling step or to the preparation or the mixture.

According to an embodiment, the target nucleic acid detection reaction may be conducted together with the detection of the endogenous internal control.

According to another embodiment, the target nucleic acid detection reaction may be conducted together with the detection of the exogenous internal control.

According to a further embodiment, the target nucleic acid detection reaction may be conducted together with the detection of the endogenous internal control and the exogenous internal control.

According to an embodiment, the internal control may be MS2 phage particle.

According to an embodiment, the internal control may be RNase P.

According to an embodiment, the internal control may be a combination of MS2 phage particle and RNase P.

According to an embodiment, the real-time nucleic acid amplification reaction using the preparation exhibits a sensitivity of 10-1000, 10-900, 10-800, 10-700, 10-600, 10-500, 10-400, 10-300, 10-200, or 10-100 copies/reaction. In detail, the sensitivity mentioned herein is based on that obtained with 20-30 μl of a real-time nucleic acid amplification reaction solution.

In an embodiment, the real-time nucleic acid amplification reaction using the preparation yields a sensitivity of 10-100 copies/reaction.

In the present disclosure, the target nucleic acid detecting composition may optionally comprise reagents necessary for a target amplification reaction (e.g., PCR), such as a nucleic acid polymerase, a buffer, a polymerase cofactor, and deoxyribonucleotide-5-triphosphates. Optionally, the target nucleic acid detecting composition may also various polynucleotide molecules, reverse transcriptase, various buffers and reagents, and an antibody inhibitory of a nucleic acid polymerase activity. In addition, the nucleic acid detecting composition may contain an oligonucleotide or reagent necessary for a positive control reaction. Optimal amounts of reagents used in a specific reaction can be easily determined by a person skilled in the art who understands the advantages of the present disclosure. Components of the nucleic acid detecting composition may exist in respective individual containers or may be contained in combination in a single container.

Unless otherwise defined, all technical and scientific terms used herein have the same meaning as commonly understood by one of ordinary skill in the art.

Features and advantages of the present disclosure are summarized as follows:

(a) The present disclosure is to provide a preparation by incubating a viscous specimen for a time allowed to prevent a target nucleic acid of the specimen from being degraded, that is, for 1 minute to 25 minutes at a temperature ensuring a lysate of the specimen, that is at 95° C. to 100° C.

(b) According to an embodiment, conventional nucleic acid extraction processes performed in many steps can be omitted, whereby the shortage of nucleic acid extraction reagents can be solved and a preparation for detecting target nucleic acid sequence in a specimen can be supplied in an inexpensive and simple manner.

Hereinafter, the present disclosure will be described in detail through examples. It would be obvious to those skilled in the art that these examples are intended to be more concretely illustrative and the scope of the present invention as set forth in the appended claims is not limited to or by the examples.

### EXAMPLES

Using preparations provided according to the present disclosure, the possibility of detecting target nucleic acid sequences in specimens was analyzed.

### Example 1

### Production of Specimens

From strain banks, SARS-CoV-2, human respiratory syncytial virus A, human respiratory syncytial virus B, influenza B virus (Yamagata), influenza A H1N1 virus, influenza A H1N1pdm virus, and influenza A H3N2 virus were purchased as summarized in Table 1.

TABLE 1
Strains

| No. | Organism | Source | Cat. No. | Strain | Status | Type |
|---|---|---|---|---|---|---|
| 1 | SARS-CoV-2 | BEI | NR-52287 | USA-WA1/2020 | Cell | RNA |
| | | | | | | virus |
| 2 | Human | Zeptometrix | 0810040ACF | 2006 Isolate | Cell | RNA |
| | respiratory | | | | | virus |
| | syncytial | | | | | |
| | virus A | | | | | |
| 3 | Human | Zeptometrix | 0810040CF | CH93(18)-18 | Cell | RNA |
| | respiratory | | | | | virus |
| | syncytial | | | | | |
| | virus B | | | | | |
| 4 | Influenza B | Zeptometrix | 0810255CF | Florida/04/06 | Cell | RNA |
| | Virus | | | | | virus |
| | (Yamagata) | | | | | |
| 5 | Influenza A | Zeptometrix | 0810036CF | New Cal/20/99 | Cell | RNA |
| | H1N1 Virus | | | | | virus |
| 6 | Influenza A | Zeptometrix | 0810165CF | California/07/09 | Cell | RNA |
| | H1N1pdm | | | | | virus |
| | Virus | | | | | |
| 7 | Influenza A | Zeptometrix | 0810240CF | Victoria/361/11 | Cell | RNA |
| | H3N2 Virus | | | | | virus |

(1-1) Production of Swab Specimens

Each of the strains was diluted in a negative swab specimen to prepare swab specimens at the concentrations indicated in Table 2, below, followed by storage at −80° C.

For the negative swab specimens, nasopharyngeal swabs and oropharyngeal (throat) swabs were each taken using the specimen collection kit CTM (Clinical Virus Transport Medium) (Noble Bio, UTNFS-3B-2N1P) and were dissolved together in CTM before storage at −80° C.

TABLE 2
Swab Specimen Concentration

| | | Test Concentration (TCID₅₀/mL) | | |
|---|---|---|---|---|
| No. | Organism | Fold 1 | Fold 2 | Fold 3 |
| 1 | SARS-CoV-2 | 3.5 × 10⁰ | 1.8 × 10⁰ | 8.8 × 10⁻¹ |
| 2 | Human respiratory | 2.63 × 10¹ | 1.31 × 10¹ | 6.55 × 10⁰ |
| | syncytial virus A | | | |
| 3 | Human respiratory | 1.05 × 10¹ | 5.25 × 10⁰ | 2.63 × 10⁰ |
| | syncytial virus B | | | |
| 4 | Influenza B Virus | 1.58 × 10⁰ | 7.88 × 10⁻¹ | 3.94 × 10⁻¹ |
| 5 | Influenza A | 5.71 × 10¹ | 2.86 × 10¹ | 1.43 × 10¹ |
| | H1N1 Virus | | | |
| 6 | Influenza A | 5.21 × 10⁰ | 2.61 × 10⁰ | 1.30 × 10⁰ |
| | H1N1pdm Virus | | | |
| 7 | Influenza A | 1.75 × 10⁰ | 8.80 × 10⁻¹ | 4.38 × 10⁻¹ |
| | H3N2 Virus | | | |

(1-2) Production of Saliva Specimen

The strains were diluted in negative saliva specimens to prepare saliva specimens at the concentrations indicated in Table 3, below, followed by storage at −20° C.

From a subject who had neither ingested any diets including water nor smoked or bushed teeth for one hours before specimen sampling, a negative saliva specimen was taken into an empty tube without bubbles and then stored at −20° C.

TABLE 3
Saliva Specimen Concentration

| | | Test Concentration (TCID₅₀/mL) | | |
|---|---|---|---|---|
| No. | Organism | Fold 1 | Fold 2 | Fold 3 |
| 1 | SARS-CoV-2 | 3.5 × 10⁰ | 1.8 × 10⁰ | 8.8 × 10⁻¹ |
| 2 | Human respiratory | 5.25 × 10¹ | 2.63 × 10¹ | — |
| | syncytial virus A | | | |
| 3 | Human respiratory | 5.21 × 10⁰ | 2.61 × 10⁰ | — |
| | syncytial virus B | | | |
| 4 | Influenza B Virus | 7.88 × 10⁻¹ | 3.94 × 10⁻¹ | — |
| | (Yamagata) | | | |
| 5 | Influenza A H1N1 | 5.71 × 10¹ | 2.86 × 10¹ | — |
| | Virus | | | |
| 6 | Influenza A H1N1pdm | 2.61 × 10⁰ | 1.30 × 10⁰ | — |
| | Virus | | | |
| 7 | Influenza A H3N2 | 1.76 × 10⁰ | 8.81 × 10⁻¹ | — |
| | Virus | | | |

### Example 2

### Heating Preparation for Detection of Target Nucleic Acid Sequence

(2-1) Swab Heating Preparations

After the specimens stored at −80° C. were thawed at room temperature, 15 μl of the transport medium containing the specimens was added to each tube and diluted with 45 μl of distilled water. Subsequently, the dilution was positioned on a real-time thermocycler (CFX96, Bio-Rad) and incubated at 98° C. for 3 minutes, followed by cooling at 4° C. for 5 minutes to give preparations.

(2-2) Saliva Heating Preparations

After the specimens stored at −20° C. were thawed at room temperature, 30 μl of 1×TE buffer (1 mM EDTA, 10 mM Tris-HCl (pH 8.0)) as a saliva transport medium was dispensed to each tube and added with 10 μl of saliva. Then, 5 μl of proteinase K was added to each tube so that proteinase K had a concentration of 2.22 mg/mL in a final volume of 45 μl. The samples thus obtained were positioned on a real-time thermocycler (CFX96, Bio-Rad) and incubated at 98° C. for 20 minutes and cooled at 4° C. for 5 minutes to give preparations.

### Example 3

### One-Step RT-PCR

Using components of the multiple One-step RT-PCR product AllPLEX™ SARS-CoV-2/FluA/FluB/RSV Assay (Cat.No RV10259X, Seegene Inc.), which can detect multiple targets in a single tube, One-step RT-PCR was performed on the heating preparations prepared in Example 2. To this end, reaction mixtures were prepared as follows.

(3-1) Reaction Mixture for Swab Heating Preparation

A total of 21 swab heating preparations obtained according to 3 concentrations of 7 strains in Example 2 were each put in an amount of 5 μl into respective tubes to which 5 μl of SC2FabR MOM, 4 μl of RNase-free Water, 1 μl of RP-V IC 2, and 5 μl of EM8 were added to afford a reaction mixture having a final volume of 20 μl.

(3-2) Reaction Mixture for Saliva Heating Preparation

A total of 15 saliva heating preparations obtained according to 2 concentrations (3 concentrations for SARS-CoV 2) of 7 strains in Example 2 were each put in an amount of 5 μl into respective tubes to which 5 μl of SC2FabR MOM, 4 μl of RNase-free Water, 1 μl of RP-V IC 2, and 5 μl of EM8 were added to afford a reaction mixture having a final volume of 20 μl.

The tubes containing the reaction mixtures prepared in (3-1) and (3-2) were positioned on a real-time thermocycler (CFX96, Bio-Rad), incubated at 50° C. for 20 minutes, and then denatured at 95° C. for 15 minutes before 3 thermal cycles of 95° C. for 10 seconds, 60° C. for 40 seconds, and 72° C. for 20 second and then 42 cycles of 95° C. for 10 seconds, 60° C. for 15 seconds, and 72° C. for 10 seconds. Signal detection was made at 60° C. and 72° C. every cycle. Using the signals detected at the temperatures, Cₜ values were analyzed. For each preparation, 24 rounds of the experiments were repeated to obtain average Cₜ values.

As a result, it was found that respiratory viruses can be detected using the swab heating preparations and saliva heating preparations provided according to the present disclosure, as is understood from the data of Tables 4 and 5. Moreover, the detection could be achieved with excellent sensitivity (LoD).

In addition, endogenous internal controls were also observed to be detectable using the preparations prepared according to the present disclosure.

TABLE 4
Average Cₜ Values and LoD of Swab Heating
Preparations According to Concentration

| | |
|---|---|
| | Avg. Cₜ Value by |
| Limit of | Concentration |

| Detection | Detection | Conc. | Avg. |
|---|---|---|---|
| target | (TCID₅₀/mL) | (TCID₅₀/mL) | Cₜ value |
| S gene | 1.8 × 10⁰ | 3.5 × 10⁰ | 31.82 |
| | | 1.8 × 10⁰ | 32.85 |
| | | 8.8 × 10⁻¹ | — |
| RdRP gene | 1.8 × 10⁰ | 3.5 × 10⁰ | 31.65 |
| | | 1.8 × 10⁰ | 33.18 |
| | | 8.8 × 10⁻¹ | — |
| N gene | 1.8 × 10⁰ | 3.5 × 10⁰ | 31.83 |
| | | 1.8 × 10⁰ | 33.52 |
| | | 8.8 × 10⁻¹ | — |
| SARS-CoV-2* | 4.4 × 10⁻¹ | — | — |
| RSV A | 1.31 × 10¹ | 2.63 × 10¹ | 33.46 |
| | | 1.31 × 10¹ | 35.42 |
| | | 6.55 × 10⁰ | — |
| RSV B | 5.25 × 10⁰ | 1.05 × 10¹ | 32.71 |
| | | 5.25 × 10⁰ | 33.91 |
| | | 2.63 × 10⁰ | — |
| Flu B | 7.88 × 10⁻¹ | 1.58 × 10⁰ | 31.70 |
| | | 7.88 × 10⁻¹ | 32.67 |
| | | 3.94 × 10⁻¹ | — |
| Flu A H1N1 | 2.86 × 10¹ | 5.71 × 10¹ | 33.04 |
| | | 2.86 × 10¹ | 33.77 |
| | | 1.43 × 10¹ | — |
| Flu A H1N1 | 2.61 × 10⁰ | 5.21 × 10⁰ | 32.81 |
| pdm | | 2.61 × 10⁰ | 34.08 |
| | | 1.30 × 10⁰ | — |
| Flu A H3N2 | 8.80 × 10⁻¹ | 1.75 × 10⁰ | 32.95 |
| | | 8.80 × 10⁻¹ | 33.92 |
| | | 4.38 × 10⁻¹ | — |

*indicating detection of at least one of the SARS-CoV-2 target genes: S gene, RdRP gene, and N gene

TABLE 5
Average Cₜ Values and LoD of Saliva Heating
Preparations According to Concentration

| | |
|---|---|
| | Avg. Cₜ Value by |
| Limit of | Concentration |

| Detection | Detection | Conc. | Avg. |
|---|---|---|---|
| target | (TCID₅₀/mL) | (TCID₅₀/mL) | Cₜ value |
| S gene | 3.5 × 10⁰ | 3.5 × 10⁰ | 34.35 |
| | | 1.8 × 10⁰ | — |
| | | 8.8 × 10⁻¹ | — |
| RdRP gene | 3.5 × 10⁰ | 3.5 × 10⁰ | 34.62 |
| | | 1.8 × 10⁰ | — |
| | | 8.8 × 10⁻¹ | — |
| N gene | 3.5 × 10⁰ | 3.5 × 10⁰ | 31.81 |
| | | 1.8 × 10⁰ | — |
| | | 8.8 × 10⁻¹ | — |
| SARS-CoV-2* | 1.8 × 10⁰ | — | — |
| RSV A | 5.25 × 10¹ | 5.25 × 10¹ | 34.45 |
| | | 2.63 × 10¹ | — |
| RSV B | 5.21 × 10⁰ | 5.21 × 10⁰ | 34.67 |
| | | 2.61 × 10⁰ | — |
| Flu B | 7.88 × 10⁻¹ | 7.88 × 10⁻¹ | 33.89 |
| | | 3.94 × 10⁻¹ | — |
| Flu A H1N1 | 5.71 × 10¹ | 5.71 × 10¹ | 33.92 |
| | | 2.86 × 10¹ | — |
| Flu A H1N1 | 2.61 × 10⁰ | 2.61 × 10⁰ | 33.84 |
| pdm | | 1.30 × 10⁰ | — |
| Flu A H3N2 | 1.76 × 10⁰ | 1.76 × 10⁰ | 34.03 |
| | | 8.81 × 10⁻¹ | — |

*indicating detection of at least one of the SARS-CoV-2 target genes: S gene, RdRP gene, and N gene

### Example 4

### Assay for Clinical Sensitivity and Clinical Specificity

Clinical sensitivity and clinical specificity were assayed between the nucleic acid extraction preparations that had undergone a nucleic acid extraction step and the swab heating preparations prepared according to the present disclosure and subsequently between the swab heating preparations and the saliva heating preparations.

(4-1) Production of Specimen

As swab clinical specimens of SARS-CoV-2, Among swab positive clinical specimens of SARS-CoV-2, 30 were selected randomly. Among swab negative clinical specimens of SARS-CoV-2, 50 were randomly selected. For saliva clinical specimens of SARS-CoV-2, 30 saliva positive clinical specimens and 50 saliva negative clinical specimens were also selected randomly.

In the case of Flu A, Flu B, and RSV clinical specimens, their positive specimens were difficult to acquire. In this regard, RSV A (Cat. No. 0810040ACF, Zeptometrix), Flu B (Cat. No. 0810255CF, Zeptometrix), and Flu A (Cat. No. 0810036CF, Zeptometrix) strains were each added to negative specimens in the same manner as in Example 1 to acquire 14 swab Flu A-positive specimens, 14 swab Flu B-positive specimens, 14 swab RSV-positive specimens, 14 saliva Flu A-positive specimens, 14 saliva Flu B-positive specimens, and 14 saliva RSV-positive specimens 14. In addition, random selection was made of 30 swab negative specimens and 30 saliva negative specimens.

(4-2) Production of Preparation for Detection of Target Nucleic Acid Sequence

Using the specimens of (4-1), swab heating preparations and saliva heating preparations were prepared in the same manner as in Example 2.

For nucleic acid extraction preparations, a nucleic acid was extracted from each of the specimens of (4-1) by using MagNA Pure 96 (Roche Diagnostics) and extraction was performed using 100 μl of an elution buffer for 200 μl of the extraction sample in the final extraction step.

(4-3) One-Step RT-PCR

One-step RT-PCR for the swab heating preparations and saliva heating preparations prepared in Example (4-2) was carried out in the same manner as in Example 3, with the exception of using the preparations of Example (4-2) instead of the preparations of Example 2.

For One-step RT-PCT for the nucleic acid extraction preparations, 10 μl of each of the nucleic acid extraction preparations was input into each tube to which 5 μl of SC2FabR MOM and 5 μl of EM8 were added to give a reaction mixture having a final volume of 20 μl.

Subsequently, each tube containing the reaction mixture was put on a real-time thermocycler (CFX96, Bio-Rad), incubated at 50° C. for 20 minutes, and denatured at 95° C. for 15 minutes, followed by 3 thermal cycles of 95° C. for 10 seconds, 60° C. for 40 seconds, and 72° C. for 20 seconds and then 42 cycles of 95° C. for 10 seconds, 60° C. for 15 seconds, and 72° C. for 10 seconds. Signal detection was made at 60° C. and 72° C. every cycle. Using the signals detected at the temperatures, Cₜ values were analyzed.

As a result, it was found that RT-PCR using the swab heating preparations exhibited superb clinical sensitivity and specificity, compared to RT-PCR using nucleic acid extraction preparations, as is understood from the data of Table 6.

In addition, as can be seen from data of Table 7, RT-PCR using the saliva heating preparations exhibited 100% clinical sensitivity and clinical specificity, relative to the swab heating preparations.

TABLE 6
Clinical Sensitivity and Specificity of Swab Heating Preparation

| | Result | | |
|---|---|---|---|
| Target | ORA* | PPA** | NPA*** |
| SARS- | 95.30% | 87.50% | 100.00% |
| CoV-2 | (95% CI: 90.56% | (95% CI: 75.93% | (95% CI: 96.11% |
| | to 98.09%) | to 94.82%) | to 100.00%) |
| RSV | 98.66% | 92.00% | 100.00% |
| | (95% CI: 95.24% | (95% CI: 73.97% | (95% CI: 97.07% |
| | to 99.84%) | to 99.02%) | to 100.00%) |
| Flu B | 100.00% | 100.00% | 100.00% |
| | (95% CI: 97.55% | (95% CI: 78.20% | (95% CI: 97.28% |
| | to 100.00%) | to 100.00%) | to 100.00%) |
| Flu A | 97.99% | 90.63% | 100.00% |
| | (95% CI: 94.23% | (95% CI: 74.98% | (95% CI: 96.90% |
| | to 99.58%) | to 98.02%) | to 100.00%) |

*Overall rate of agreement (ORA);
**Positive percent agreement (PPA);
***Negative percent agreement (NPA)

TABLE 7
Clinical Sensitivity and Specificity of Saliva Heating Preparation

| | Results | | |
|---|---|---|---|
| Target | ORA* | PPA** | NPA*** |
| SARS- | 100.00% | 100.00% | 100.00% |
| CoV-2 | (95% CI: 95.49% | (95% CI: 88.43% | (95% CI: 92.89% |
| | to 100.00%) | to 100.00%) | to 100.00%) |
| RSV | 100.00% | 100.00% | 100.00% |
| | (95% CI: 95.01% | (95% CI: 76.84% | (95% CI: 93.84% |
| | to 100.00%) | to 100.00%) | to 100.00%) |
| Flu B | 100.00% | 100.00% | 100.00% |
| | (95% CI: 95.01% | (95% CI: 76.84% | (95% CI: 93.84% |
| | to 100.00%) | to 100.00%) | to 100.00%) |
| Flu A | 100.00% | 100.00% | 100.00% |
| | (95% CI: 95.01% | (95% CI: 76.84% | (95% CI: 93.84% |
| | to 100.00%) | to 100.00%) | to 100.00%) |

*Overall rate of agreement (ORA);
**Positive percent agreement (PPA);
***Negative percent agreement (NPA)

1. A method for providing a preparation for detection of a target nucleic acid sequence in a specimen, the method comprising the steps of:
(a) incubating a specimen stored in a transport medium at 95° C. to 100° C. for 1 minute to 25 minutes to provide a preparation, wherein the specimen is lysed by the incubation and has viscosity; and
(b) mixing the preparation with a composition for detecting a target nucleic acid.

2. The method of claim 1, wherein the specimen is a swab, saliva, or a combination thereof.

3. The method of claim 2, wherein the swab is a nasopharyngeal swab, an oropharyngeal swab, a saliva swab, a genital swab, a rectal swab, or a combination of at least two thereof.

4. The method of claim 1, wherein the target nucleic acid is a viral nucleic acid.

5. The method of claim 1, wherein the target nucleic acid is an RNA viral nucleic acid.

6. The method of claim 1, wherein the transport medium is a preservative transport medium for a specimen.

7. The method of claim 1, wherein the transport medium contains no materials for lysis of a specimen.

8. The method of claim 1, wherein the incubation temperature ranges from 97° C. to 100° C.

9. The method of claim 2, wherein when the specimen is a swab, the incubation is carried out for 2 minutes to 4 minutes.

10. The method of claim 2, wherein when the specimen is saliva, the incubation is carried out for 18 minutes to 22 minutes.

11. The method of claim 1, wherein the specimen stored in the transport medium in step (a) or the preparation in step (b) is 2-fold to 5-fold diluted.

12. The method of claim 1, wherein the preparation is cooled to 2° C. to 10° C. and mixed with the composition for detecting a target nucleic acid.

13. The method of claim 1, wherein the mixture is used in the target nucleic acid detection reaction.

14. The method of claim 13, wherein the target nucleic acid detection reaction includes RT-PCR (Reverse Transcription PCR).
